(19) United States
(12) Patent Application Publication
(10) Pub. No.: US 2011/0206357 A1
(43) Pub. Date: Aug. 25, 2011

(54) CHEMICAL DELIVERY ASSEMBLY FOR COUPLING WITH AN APPLIANCE

(75) Inventors: Joe Scheiber, Kimmell, IN (US); Robert G. Cox, Kalamazoo, MI (US)

(73) Assignee: Group Dekko, Inc.

(21) Appl. No.: 13/045,565

(22) Filed: Mar. 11, 2011

Related U.S. Application Data
(62) Division of application No. 11/956,931, filed on Dec. 14, 2007.
(60) Provisional application No. 60/870,220, filed on Dec. 15, 2006.

(51) Int. Cl. A61L 9/03

(52) U.S. Cl. 392/386

(57) ABSTRACT

A chemical delivery assembly includes a volatilizable chemical member and a housing member. The volatilizable chemical member includes a volatilizable chemical. The housing member carries the volatilizable chemical member and is configured for being carried by and coupled with an appliance having an electrical supply and/or an appliance element used to controllably emit a volatilized chemical from the volatilizable chemical.

### CROSS REFERENCE TO RELATED APPLICATIONS

This is a division of U.S. patent application Ser. No. 11/956,931, entitled “CHEMICAL DELIVERY ASSEMBLY FOR COUPLING WITH AN APPLIANCE”, filed Dec. 14, 2007, which is incorporated herein by reference. U.S. patent application Ser. No. 11/956,931 is a non-provisional application based upon U.S. provisional patent application Ser. No. 60/870,220, entitled “AIR FRESHENER ASSEMBLY FOR COUPLING WITH AN APPLIANCE”, filed Dec. 15, 2006, which is incorporated herein by reference.

### BACKGROUND OF THE INVENTION

1. Field of the Invention

The present invention relates to appliance assemblies, and, more particularly, to chemical delivery assemblies.

2. Description of the Related Art

One type of chemical delivery assembly is an air freshener assembly. Air freshening is often needed in the area surrounding major appliances and/or sometimes internally within the appliance. For example, dirty clothes stored in the area surrounding a laundry washing machine create a need for air freshening, or an end-user of a laundry dryer may use a sheet or spray to deodorize the interior of the dryer. Similarly, dirty dishes contained in a dishwasher can also create a need for air freshening. However, in the areas surrounding these appliances, a shortage of accessible outlets often exists. Thus, installing a conventional direct outlet supported chemical delivery assembly, such as air freshener, is often difficult.

What is needed in the art is a chemical delivery assembly that can be mechanically and operatively incorporated into an appliance.

### SUMMARY OF THE INVENTION

The present invention provides a chemical delivery assembly that can be mechanically and operatively incorporated into an appliance.

The invention in one form is directed to an appliance assembly which includes an appliance and a chemical delivery assembly. The appliance includes an electrical supply and/or an appliance element. The chemical delivery assembly includes a volatilizable chemical and is carried by and coupled with the appliance. The appliance assembly uses the electrical supply and/or the appliance element to controllably emit a volatilized chemical from the volatilizable chemical.

The invention in another form is directed to a chemical delivery assembly which includes a volatilizable chemical member and a housing member. The volatilizable chemical member includes a volatilizable chemical. The housing member carries the volatilizable chemical member and is configured for being carried by and coupled with an appliance having an electrical supply and/or an appliance element used to controllably emit a volatilized chemical from the volatilizable chemical.

The invention in yet another form is directed to a method of using a chemical delivery assembly. The method includes the steps of providing, heating, and emitting. The providing step provides an appliance which includes an electrical supply and/or an appliance element, the chemical delivery assembly including a volatilizable chemical and being carried by and coupled with the appliance. The heating step heats the volatilizable chemical using the electrical supply and/or the appliance element. The emitting step controllably emits a volatilized chemical from the volatilizable chemical.

An advantage of the present invention is that the chemical delivery assembly is mechanically incorporated into an appliance.

Another advantage is that the chemical delivery assembly is readily accessible considering that the chemical delivery assembly can share the same space as the control panel for the appliance.

Yet another advantage is that the chemical delivery assembly can be an air freshener assembly which deodorizes the area surrounding the appliance and/or an area internal to the appliance.

Yet another advantage is that the chemical delivery assembly can receive its electric supply directly from the appliance.

Yet another advantage is that the chemical delivery assembly can emit a volatilized chemical using heat recycled from the appliance itself.

Yet another advantage is that the chemical delivery assembly can use heat from a heater within the appliance.

Yet another advantage is that the chemical delivery assembly can use incidental heat produced by one or more appliance elements which are not otherwise heaters.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a perspective view of the appliance assembly according to the present invention, the chemical delivery assembly being installed in the appliance;

FIG. 2 is a perspective view of the appliance assembly of FIG. 1, the chemical delivery assembly exploded from the appliance;

FIG. 3 is a schematic diagram showing the appliance of FIG. 1 electrically coupled with an external power source, as well as the appliance and the chemical delivery assembly mechanically connected together;

FIG. 4 is an exploded, perspective view of the chemical delivery assembly of FIG. 1 broken away from the appliance;

FIG. 5 is a perspective view of the chemical delivery assembly of FIG. 1;

FIG. 6 is an exploded, perspective view of the chemical delivery assembly of FIG. 1 broken away from the appliance;

FIG. 7 is an exploded, perspective view of the chemical delivery assembly of FIG. 1 broken away from the appliance;

FIG. 8 is a schematic diagram showing another embodiment of the appliance assembly according to the present invention; and

FIG. 9 is a schematic diagram showing yet another embodiment of the appliance assembly according to the present invention

Corresponding reference characters indicate corresponding parts throughout the several views. The exemplifications set out herein illustrate embodiments of the invention, and such exemplifications are not to be construed as limiting the scope of the invention in any manner.

### DETAILED DESCRIPTION OF THE INVENTION

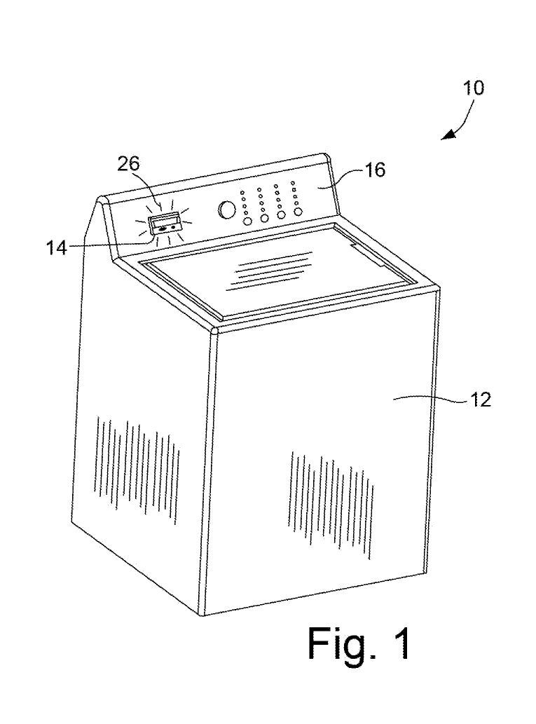

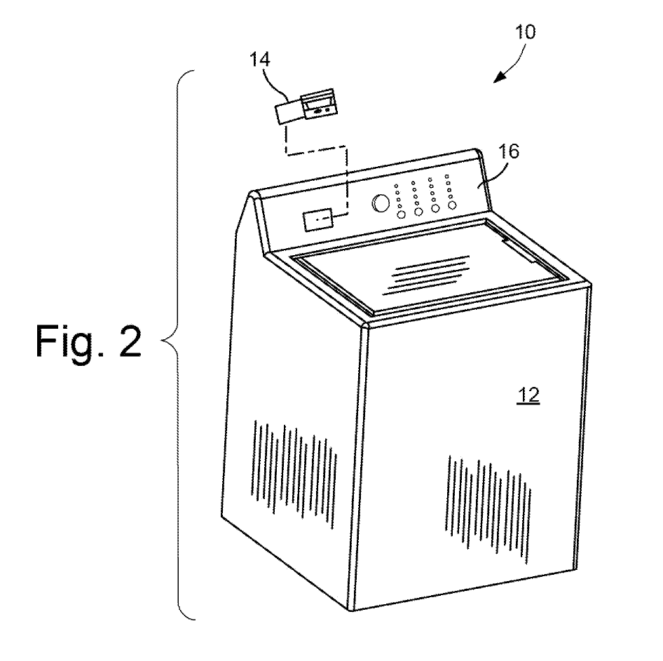

Referring now to the drawings, and more particularly to FIGS. 1 and 2, there is shown an appliance assembly 10 which generally includes an appliance 12 and a chemical delivery assembly 14 carried by and coupled with appliance 12. Appliance 12 can include an electrical supply and/or an appliance element 13.

Appliance 12 can be, for example, a laundry washer 12 (as shown in FIGS. 1-2), a laundry dryer, a dishwasher, a refrigerator, an oven (such as a range oven or a microwave oven), a stove, and/or a television. This listing of appliances is not intended to be limiting. Appliance 12, such as laundry washing machine 12, can include a control panel 16 to which chemical delivery assembly 14 can be attached; that is, chemical delivery assembly 14 can be fitted in a slot formed in control panel 16, as shown in FIGS. 1 and 2. Control panel 16 of washing machine 12 can include operator controls related to water temperature, volume of water, power on/off, and washing cycles. While this detailed description primarily refers to appliance 12 and laundry washing machine 12, it is understood that a different appliance could be used.

Chemical delivery assembly 14 can be an air freshener/deodorizer assembly, an air cleaner assembly, a bug repellant assembly, and/or a vaporizer for, for instance, providing medicinal vapors. This listing of applications of chemical delivery assembly 14 is not intended to be limiting. For simplicity, this detailed description primarily refers to chemical delivery assembly 14 as an air freshener assembly 14, but chemical delivery assembly 14 does not have to be an air freshener assembly.

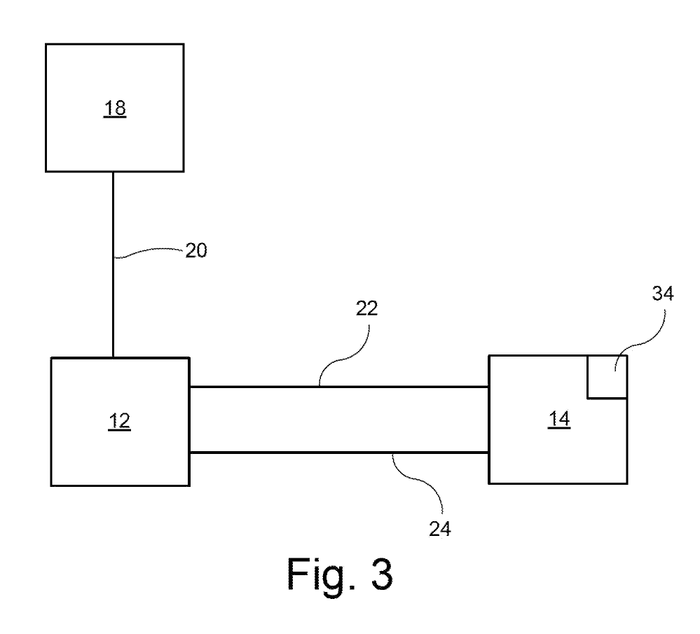

FIG. 3 shows that appliance 12 includes an electrical supply that can be provided to chemical delivery assembly 14. More specifically, FIG. 3 shows that appliance 12 can receive its electrical supply from a power source 18 which is external to appliance 12, as illustrated by line 20 in FIG. 3. Further, FIG. 3 shows that appliance 12 can supply electrical supply received from external power source 18 to chemical delivery assembly 14, as illustrated by line 22 in FIG. 3. Further, FIG. 3 shows that appliance 12 is mechanically connected to (i.e., carries, coupled with) chemical delivery assembly 14, as illustrated by line 24 in FIG. 3. FIG. 3 also shows that chemical delivery assembly 14 can include at least one heating element 34.

External power source 18 can be a power plant 18 which supplies electricity (also referred to as “electrical supply”) to a building in which appliance 12 is located. In such a scenario, chemical delivery assembly 14 is mechanically and electrically coupled with appliance 12, which is, for example, plugged into a conventional wall outlet of the building in which appliance 12 is located. Power plant 18 supplies electrical supply to the building, which in turn supplies electrical supply to appliance 12 (via the electrical wiring in the building), which in turn supplies electrical supply to chemical delivery assembly 14 (via the electrical wiring of appliance 12). As such, electrical supply included in appliance 12 can come from power plant 18 and be routed to and through appliance 12 to chemical delivery assembly 14, as shown in FIG. 3. It is to be noted that appliance 12 need not itself be “running” for chemical delivery assembly 14 to be empowered with electricity and, thus, to function. That is, washing machine 12 need not be turned on and washing clothes, for instance, for air freshener assembly 14 to receive electrical supply from washing machine 12. As such, electrical supply can come from power source 18 external to washing machine 12, and electrical supply can be routed through appliance 12 to air freshener assembly 14, for example, by wiring the washing features of washing machine 12 and air freshener assembly 14 in parallel to one another. Alternatively, solar energy or chemical energy (i.e., fuel cell) can be converted to electrical energy/supply which is used by appliance 12. Alternatively (not shown), appliance 12 can supply its own electrical supply, such as by using batteries; that electrical supply can then be supplied to chemical delivery assembly 14.

Chemical delivery assembly 14 includes a volatilizable chemical 46 and can be carried by and coupled with appliance 12. Appliance assembly 10 uses electrical supply of appliance 12 and/or appliance element 13 to controllably emit a volatilized chemical 26 from volatilizable chemical 46. More specifically, chemical delivery assembly 14 can include a volatilizable chemical member 30 and a housing member 28. Volatilizable chemical member 30 includes volatilizable chemical 46. Housing member 28 carries volatilizable chemical member 30 and can be carried by and coupled with appliance 12. Appliance 12 has electrical supply and/or appliance 13 (usually, appliance 12 has both electrical supply and appliance element 13. Electrical supply and/or appliance 13 can be used to controllably emit volatilized chemical 26 from volatilizable chemical 46. That is, both electrical supply and appliance element 13 can be used to controllably emit volatilized chemical 26; alternatively, either electrical supply or appliance element 13 (not both) can be used to controllably emit volatilized chemical 26.

The following describes three embodiments of the present invention. The first embodiment provides that electrical supply of appliance 12 is provided to chemical delivery assembly 14 and used to heat a heating element 34 of chemical delivery assembly 14, heating element 34 being used to heat volatizible chemical 46 to controllably emit volatized chemical 26. As described below, heating element 34 of chemical delivery assembly 14 is electrically coupled with electrical supply of appliance 12, in the first embodiment. The second and third embodiments of the present invention provide that chemical delivery assembly 14 does not have its own heating element (i.e., heating element 34), although chemical delivery assembly 14 can have its own heating element in the second and third embodiments. As such, in the second and third embodiments chemical delivery assembly 14 is not necessarily electrically coupled with electrical supply of appliance 12, considering that heating element 34 of chemical delivery assembly 14 is not being used to volatize volatizible chemical 46 (although, chemical delivery assembly 14 could be electrically coupled with electrical supply of appliance 14 for purposes other than volatizing volatizible chemical 46). Rather, in the second and third embodiments volatizible chemical 46 of chemical delivery assembly 14 is heated by appliance element 13 (more than one appliance element 13 can be used to so heat volatizible chemical 46), which is not chemical delivery assembly 14. Appliance element 13 refers to an element coupled with appliance 12 (i.e., in appliance 12) which radiates or produces heat (i.e., a heater, a motor, a compressor, a light). The second embodiment, more specifically, provides that appliance element 13 is itself a heater. On the other hand, the third embodiment provides that appliance element 13 is not a heater but is another appliance element, such as a motor, a compressor, or a light, which produces merely incidental heat; that incidental heat, from one or more such appliance elements 13, is then used to heat volatizible chemical 46 to controllably emit volatized chemical 46 therefrom. The following describes these three embodiments in more detail.

Regarding the first embodiment of the present invention, chemical delivery assembly 14 can be air freshener assembly 14, which is carried by and coupled with appliance 12. As such, volatilized chemical 26, volatilizable chemical member 30, and volatilizable chemical 46 are respectively a fragrance 26, a fragrance member 30, and a fragrance gel 46 (that is, volatilizable chemical 46 is formed as a fragrance gel 46). FIG. 1 shows fragrance 26 as a plurality of small lines generally projecting from around air freshener assembly 14.

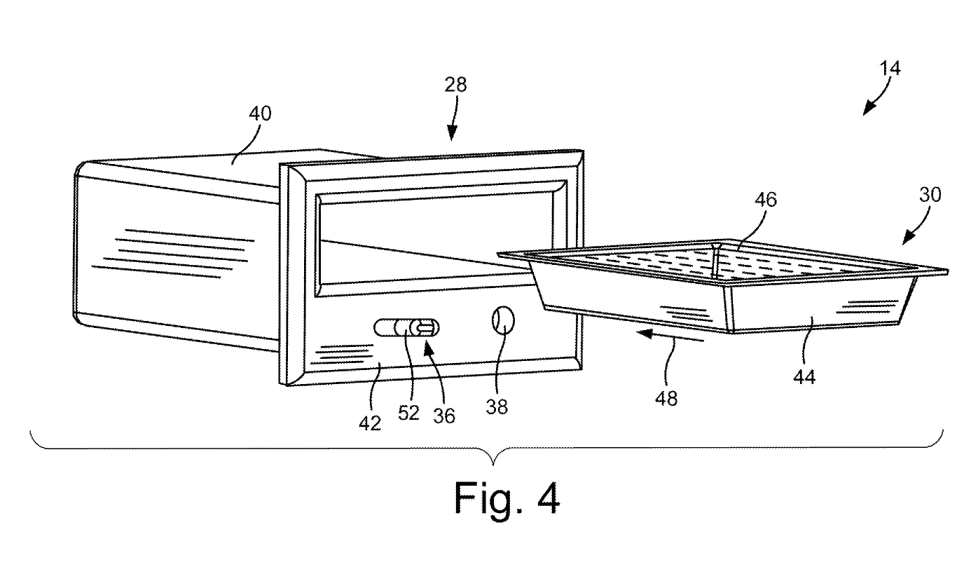

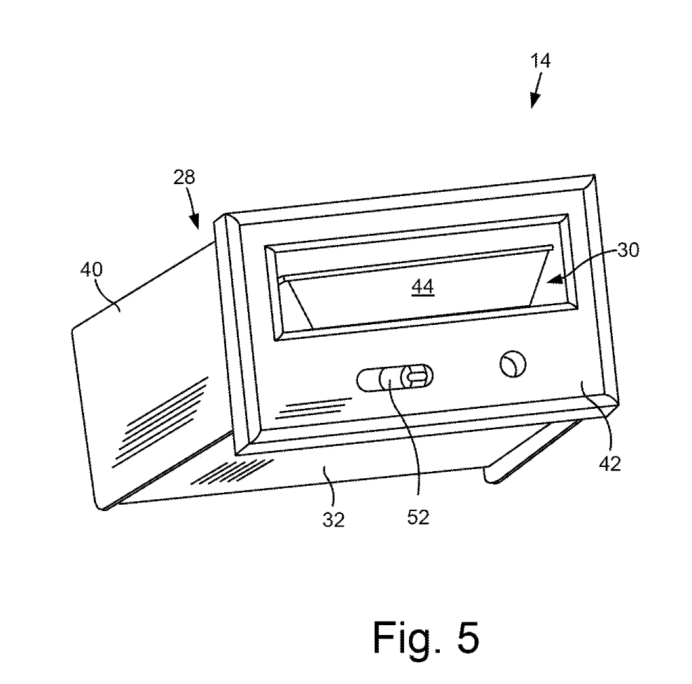

FIGS. 4-7 show that air freshener assembly 14 can include housing member 28, fragrance member 30, a printed circuit board 32 (which can be a printed control wire board), at least one heating element 34, a switch assembly 36, and an indicator light 38. Housing member 28 is carried by and coupled with, and thereby attached to, appliance 12 (as shown in FIGS. 1-3). Housing member 28 can be attached to appliance 12 in any suitable manner, such as by using brackets, bolts, screws, rivets, and/or snap-fit features. Housing member 28 can include a generally box-shaped body 40 with a front face plate 42. Housed within body 40 and front face plate 42 are fragrance member 30 (also referred to as volatilizable chemical member 30), printed circuit board 32, and at least one heating element 34. Housing member 28 carries fragrance member 30.

Fragrance member 30 (also referred to as volatilizable chemical member 30) includes a fragrance tray 44 which is slidably received by housing member 28 and a fragrance gel 46 which is carried by fragrance tray 44. Fragrance gel 46 is shown only in FIG. 4. The lead line for reference number 46 touches the surface of gel 46, the surface of gel 46 being shown as a solid line and the remainder of gel 46 being shown in broken lines. Fragrance tray 44 can be inserted into a cavity of housing member 28 (in the direction of arrow 48), via a cutout in front face plate 42, by an end-user. Fragrance tray 44 can also be removed from housing member 28 (in the direction opposite arrow 48) by the end-user when, for example, the life of fragrance gel 46 has expired (i.e., when fragrance gel 46 no longer emits fragrance 26). Fragrance tray 44 can include a bottom wall, four side walls extending generally upwardly from the bottom wall and generally forming a well with an open top, and flanges or top edges extending laterally from the top section of each of the four side walls. The top edges can extend generally parallel to the bottom wall of fragrance tray 44, as shown in FIGS. 4-7. A semi-permeable membrane or liner (not shown) can be adhered or otherwise attached to the top edges of fragrance tray 44 (i.e., attached to the laterally extending flanges), the membrane being generally parallel with the bottom wall of fragrance tray 44 and covering the open portion of fragrance tray 44. Contained in the well of fragrance tray 44 and underneath the membrane is fragrance gel 46, as shown in FIG. 4. The membrane is configured to permit small particles from fragrance gel 46 (when fragrance gel 46 is heated and volatilized) to pass through the membrane and to disperse into the air surrounding air freshener assembly 14 and appliance 12 and/or to disperse internally within appliance 12. When fragrance tray 44 is full, fragrance gel 46 can have a top surface which comes up to a generally horizontal level in fragrance tray 44 just below the plane formed by the top edges of fragrance tray 44. The membrane can be almost completely transparent, which can permit an end-user of air freshener assembly 14 to view whether fragrance tray 44 is empty of fragrance gel 46 and in need of replacing.

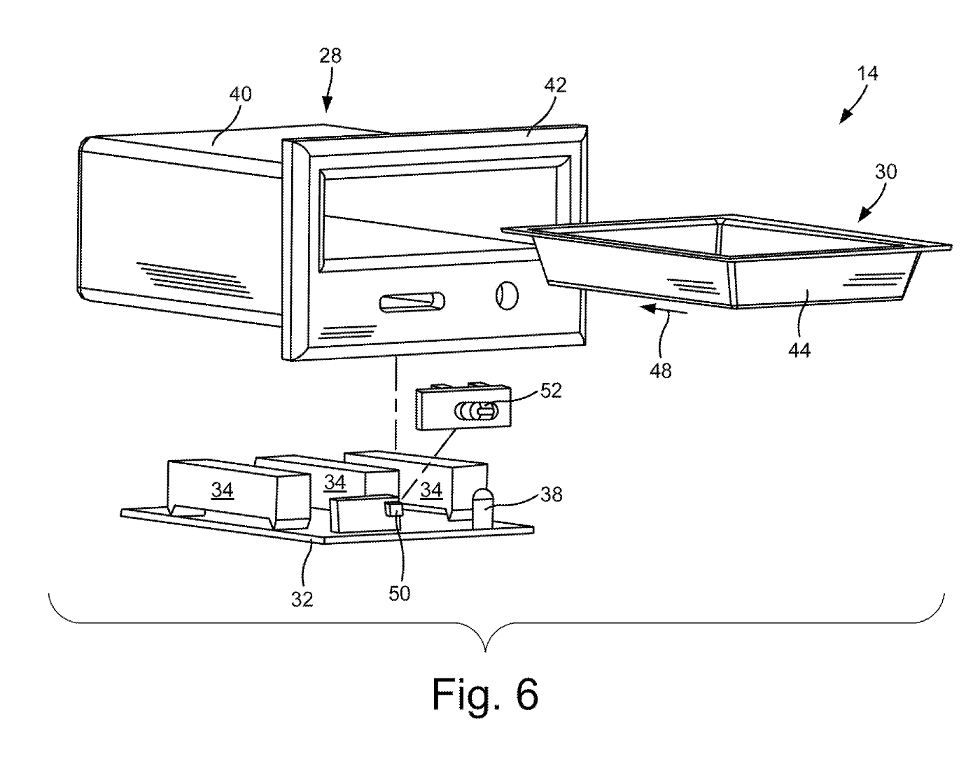

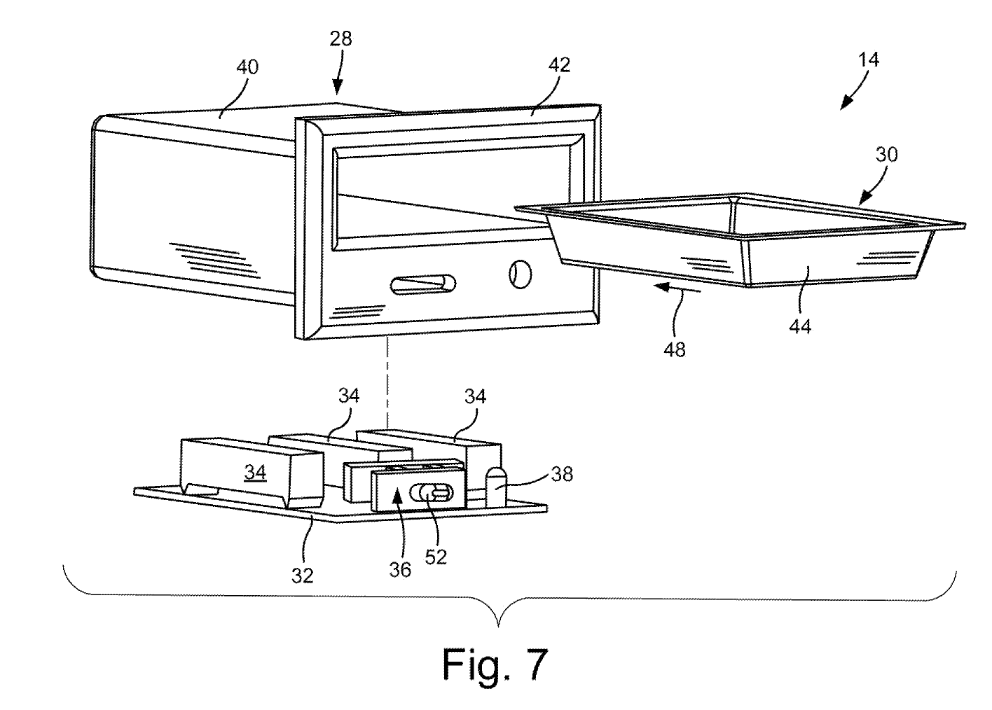

Printed circuit board 32 can be coupled with housing member 28 and positioned below fragrance tray 44. Printed circuit board 32 can form a mounting platform for various elements housed within housing member 28. For instance, electrical circuitry can be mounted to or otherwise formed on printed circuit board 32 so as to electrically interconnect electrical circuitry of appliance 12 with pertinent elements of air freshener assembly 14, such as one or more heating elements 34 and/or indicator light 38. The electrical circuitry of air freshener assembly 14 (such as electrical circuitry mounted to printed circuit board 32) can be coupled with the electrical circuitry of appliance 12 in any suitable manner (as indicated in FIG. 3), such as, for instance, hardwiring the electrical circuitry of air freshener assembly 14 with the electrical circuitry of appliance 12 or coupling the electrical circuitry of air freshener assembly 14 with the electrical circuitry of appliance 12 using electrical connectors (such as a plug). FIGS. 6 and 7 show printed circuit board exploded from housing member.

Heating element 34 can include a plurality of heating elements 34. FIGS. 6 and 7 show three heating elements 34 mounted to printed circuit board 32. As such, heating elements 34 can be coupled with housing member 28 by being mounted on printed circuit board 32 under fragrance tray 44, and printed circuit board 32 can be directly attached to housing member 28. Heating element 34 can receive electrical supply from appliance 12 and thereby heat fragrance member 30 in order to emit fragrance 26 therefrom. That is, heating element 34 is heated using electrical supply from appliance 12. Stated another way, upon receiving—and thereby being heated using—electrical supply from appliance 12, heating element 34 heats fragrance gel 46 to controllably emit fragrance 26. Electrical circuitry can be formed on printed circuit board and routed to and from heating element 34. Upon receiving electrical supply from appliance 12, heating elements 34 themselves heat up and, given thermal communication between heating elements 34 and fragrance gel 46, thereby heat up fragrance gel 46. Thus, by being positioned under fragrance tray 44 and electrically coupled with appliance 12, heating elements 34 heat fragrance gel 46, using electrical supply from appliance 12, so that fragrance gel 46 controllably emits fragrance 26.

Switch assembly 36 can include a switch 50 and a switch actuator 52. Switch 50 can be mounted to printed circuit board 32 and electrically coupled with appliance 12, heating elements 34, and indicator light 38. Switch 50 can include an “off” setting and a plurality of “on” settings including low, medium, and high settings corresponding to low, medium, and high—relatively speaking—amounts of electricity supplied from appliance 12 to heating elements 34. Switch actuator 52 can be coupled with, such as by being mounted directly on, switch 50 and, thus, can serve as an interface between the end-user and switch 50. Switch actuator 52 can protrude through a cutout defined in front face plate 42 of housing member 28 and, thus, be visible and accessible to the end-user of air freshener assembly 14. Switch assembly 36 is, thus, configured for adjusting an amount of electrical supply provided to heating elements 34. FIG. 6 shows switch actuator 52 of switch assembly 36 exploded from switch 50, while FIG. 7 shows switch actuator 52 connected to switch 50.

Indicator light 38 is configured for indicating to an end-user whether air freshener assembly 14 is “on” or “off”. If indicator light 38 is lit, then air freshener assembly 14 is receiving electrical supply from appliance 12, such as when switch 50 is positioned in any of the “on” settings. Conversely, if indictor light 38 is not lit, then air freshener assembly 14 is not receiving electrical supply from appliance 12, such as when switch 50 is in the “off” position. Indicator light 38 can be visible to an end-user through an aperture in front face plate 42 of housing member 28, and the aperture may include a lens covering indicator light 38.

Air freshener assembly 14 controllably emits fragrance 26 in order to avoid user habituation to fragrance 26. In other words, under certain circumstances, an end-user of air freshener assembly 14 may no longer detect fragrance 26, and this condition can be referred to as “user habituation.” To avoid user habituation, one or more factors can be controllably varied. These factors can include time, temperature, and/or the number of different fragrances used. Regarding time, the turning “off” or “on”, or the setting at one or more of the “on” settings, of air freshener assembly 14 can be controllably varied relative to the day (i.e., Saturdays and Tuesdays), the time-of-day (i.e., on “high” at 11:00 a.m. and “off” at 3:00 p.m.), and/or duration (i.e., on “low” for thirty minutes followed by being on “high” for fifteen minutes). Similarly, air freshener assembly 14 can volatilize more or less fragrance 26 depending on the ambient temperature about air freshener assembly 14. Further, air freshener assembly 14 can include more than one fragrance 26 (FIG. 4 shows only one fragrance 26), and different fragrances 26 can be selectively volatilized, for instance, depending on time and/or temperature. Stated more broadly, chemical delivery assembly 14 controllably emits volatilized chemical 26 from volatilizable chemical 46 in order to avoid user-habituation to volatilized chemical 26, in whatever form the application dictates.

In use, fragrance tray 44 full of fragrance gel 46 can be slid into housing member 28 of air freshener assembly 14 in the direction of arrow 48. Switch 50, using switch actuator 52, can then be moved to one of the “on” settings, thereby enabling electricity to be supplied from appliance 12 to air freshener assembly 14. In so doing, heating elements 34 heat up and heat fragrance gel 46 so as to emit fragrance 26. Air freshener assembly 14 can be selectively turned “on” (at various settings, as already mentioned) and “off”. When air freshener assembly 14 no longer emits fragrance 26 (such as when fragrance gel 46 has dissipated) or when so desired, fragrance tray 44 can be removed from housing member 28 of air freshener assembly 14 in a direction opposite arrow 48. A new fragrance tray 44, containing additional fragrance gel 46, can then be inserted into housing member 28. Upon doing so, switch 50, using switch actuator 52, can again be selectively moved to one of the “on” settings.

According to the second embodiment of the present invention, appliance 12 includes appliance element 13 which is a heater 54. Heater 54 is configured for producing heat upon receiving electrical supply of appliance 12, heater 54 being configured for heating volatilizable chemical 46 to controllably emit volatilized chemical 26. Stated another way, volatilizable chemical member 30 is configured for receiving heat from appliance element 13 which is heater 54, heater 54 being configured for producing heat upon receiving electrical supply of appliance 12, in order to heat volatilizable chemical 46 to controllably emit volatilized chemical 26.

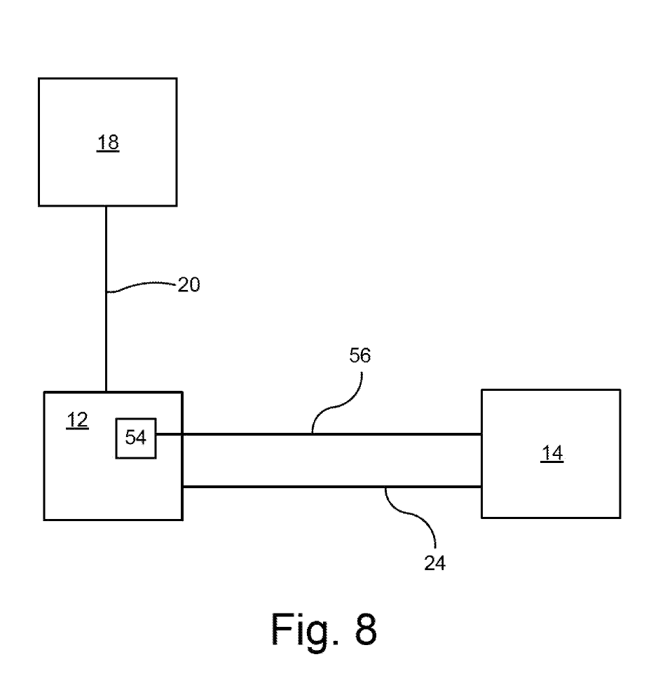

In this embodiment, heater 54 is not chemical delivery assembly 14 and is not a part of chemical delivery assembly 14. Heater 54 is not the same element as heating element 34; indeed, chemical delivery assembly 14 may not even have heating element 34 in this embodiment (but can, optionally, include heating element 34). FIG. 8 shows this embodiment schematically. In FIG. 8 are appliance 12, chemical delivery assembly 14, external power source 18, and heater 54. Power source 18 provides electrical supply to appliance 12, as illustrated by line 20 in FIG. 8. Appliance 12 includes heater 54, as illustrated by box 54 within the box for appliance 12 in FIG. 8. Appliance 12 is mechanically connected to chemical delivery assembly 14, as illustrated by line 24 in FIG. 8. Further, heater 54 is thermally connected to chemical delivery assembly 14 such that volatilizable chemical 46 is heated when heater 54 produces heat, the thermal connection between heater 54 and chemical delivery assembly 14 being illustrated by line 56 in FIG. 8. In this embodiment, electrical supply of appliance 12 may not be supplied (but can be supplied as so desired) to chemical delivery assembly 14, considering that heating element 34 (which may not even be included in chemical delivery assembly 14 in this embodiment) is not being used to heat volatilizable chemical 46. A practical example of this embodiment of the present invention is air freshener assembly 14 being carried by and coupled with a laundry dryer 12. In this example, the heater 54 used to heat the interior of dryer 12 so as to dry clothing can be in thermal communication with air freshener assembly 14 so as to heat up fragrance gel 46 (and, thus, to emit fragrance 26). Thus, when dryer 12 is running, heater 54 produces heat which is communicated to fragrance gel 46. For example, heat from heater 54 can be directly communicated to chemical delivery assembly 14 or noninsulated heat (heat that would otherwise be wasted) from heater 54 can be recycled to chemical delivery assembly 14.

According to the third embodiment of the present invention, appliance element 13 is an appliance element 58, which is not a heater. Appliance element 54 produces, upon receiving electrical supply of appliance 12, incidental heat which, at least in part, heats volatilizable chemical 46 in order to controllably emit volatilized chemical 26. Stated another way, volatilizable chemical member 30 is configured for receiving incidental heat from appliance element 58 which produces the incidental heat, upon receiving electrical supply of appliance 12, and thereby, at least in part, heats volatilizable chemical 46 to controllably emit volatilized chemical 26.

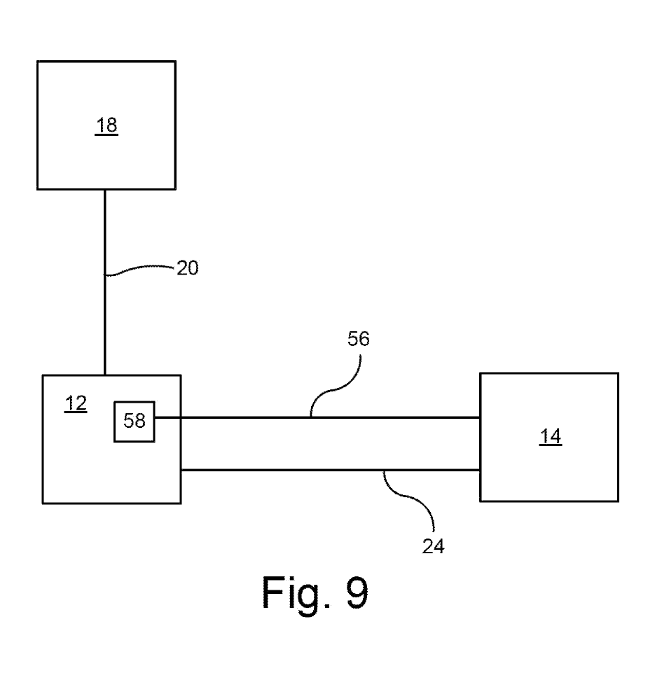

In this embodiment, appliance element 58 is not chemical delivery assembly 14 and is not a part of chemical delivery assembly 14. Appliance element 58 is not the same element as heating element 34 (nor is appliance element 58 heater 54 or any other heater); indeed, chemical delivery assembly 14 may not even have heating element 34 in this embodiment (but can, optionally, include heating element 34). FIG. 9 shows this embodiment schematically. In FIG. 9 are appliance 12, chemical delivery assembly 14, external power source 18, and appliance element 58. Power source 18 provides electrical supply to appliance 12, as illustrated by line 20 in FIG. 9. Appliance 12 includes appliance element 58, as illustrated by box 58 within the box for appliance 12 in FIG. 9. Appliance 12 is mechanically connected to chemical delivery assembly 14, as illustrated by line 24 in FIG. 9. Further, appliance element 58 is thermally connected to chemical delivery assembly 14 such that volatilizable chemical 46 is heated when appliance element 58 produces incidental heat, the thermal connection between appliance heating element 58 and chemical delivery assembly 14 being illustrated by line 56 in FIG. 9. In this embodiment, electrical supply of appliance 12 may not be supplied (but can be supplied as so desired) to chemical delivery assembly 14, considering that heating element 34 (which may not even be included in chemical delivery assembly 14 in this embodiment) is not being used to heat volatilizable chemical 46. A practical example of this embodiment of the present invention is air freshener assembly 14 being carried by and coupled with a refrigerator 12. In this example, a compressor 58 of refrigerator 12 can radiate incidental heat when compressor 58 is running. Since compressor 58 and air freshener assembly 14 are in thermal communication with each other, the incidental heat radiated by compressor 58 heats up fragrance gel 46 so as to controllably emit fragrance 26. Examples of appliance element 58 include a compressor, a motor, or a light. One or more appliance elements 58 can be used to heat, and thereby actuate, the volatizible packet of chemical delivery assembly 14. As such, the third embodiment involves recycling heat from appliance 12 itself.

Heater 54 and appliance element 58 can be distinguished from one another in that appliance element 58 merely produces incidental heat, whereas heater 54 does not produce merely incidental heat; rather, heater 54 is a heater. That is, heater 54 has the primary purpose of producing heat (in a controllable amount, for example) so as to heat something else (i.e., a room, clothes in a dryer) and is, thus, a heater. By contrast, appliance element 58 does not have the primary purpose of producing heat so as to heat something else; appliance element 58 has the primary purpose of doing something else. But, appliance element 58 does radiate incidental heat, the heat radiated being incidental to appliance element 58 accomplishing its primary purpose. This incidental heat can be described as “waste” if not otherwise used, for example, as described herein to heat volatilizable chemical 46.

The present invention further provides a method of using chemical delivery assembly 14. The method includes the steps of providing, heating, and emitting. The providing step provides appliance 12 including an electrical supply and/or appliance element 13, chemical delivery assembly 14 including volatilizable chemical 46 and being carried by and coupled with appliance 12. The heating step heats volatilizable chemical 46 using the electrical supply of appliance 12 and/or appliance element 13. The emitting step controllably emits volatilized chemical 26 from volatilizable chemical 46. The method can further include the step of supplying the electrical supply to chemical delivery assembly 14. Volatilized chemical 26 can be provided internally to appliance 12 in addition to, or instead of, being provided to an area about or surrounding appliance 12. That is, air freshener assembly 14 can be used to emit fragrance 26 internally relative to appliance 12 to directly “freshen” or deodorize an end-user's items in appliance 12; for example, laundry in a dryer 12 can be scented or deodorized using air freshener assembly 14 in conjunction with appliance 12 and, thus, without the use of a deodorizing sheet or spray. According to the second embodiment of the present invention (as mentioned already above), appliance element 13 can be heater 54 which produces heat upon receiving electrical supply of appliance 12, heater 54 heating volatilizable chemical 46 to controllably emit volatilized chemical 26. According to the third embodiment of the present invention (as mentioned already above), appliance element 13 can be appliance element 58 which produces, upon receiving electrical supply of appliance 12, incidental heat which, at least in part, heats volatilizable chemical 46 in order to controllably emit volatilized chemical 26.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A chemical delivery assembly, comprising:
a volatilizable chemical member including a volatilizable chemical; and
a housing member carrying said volatilizable chemical member and configured for being carried by and coupled with an appliance having at least one of an electrical supply and an appliance element used to controllably emit a volatilized chemical from said volatilizable chemical.

2. The chemical delivery assembly of claim 1, wherein said appliance is at least one of a laundry washer, a laundry dryer, a dishwasher, a refrigerator, an oven, and a television.

3. The chemical delivery assembly of claim 1, wherein said chemical delivery assembly is an air freshener assembly and said volatilizable chemical is formed as a fragrance gel, said volatilizable chemical member including a fragrance tray slidably received by said housing member, said fragrance gel being carried by said fragrance tray.

4. The chemical delivery assembly of claim 3, wherein said volatilized chemical is a fragrance and said air freshener assembly includes a printed circuit board and at least one heating element, said printed circuit board coupled with said housing member, said at least one heating element mounted on said printed circuit board under said fragrance tray and configured for receiving said electrical supply from said appliance and thereby heating said fragrance gel to controllably emit said fragrance.

5. The chemical delivery assembly of claim 1, wherein said volatilizable chemical member is configured for receiving heat from said appliance element which is a heater, said heater being configured for producing heat upon receiving said electrical supply in order to heat said volatilizable chemical to controllably emit said volatilized chemical.

6. The chemical delivery assembly of claim 1, wherein said volatilizable chemical member is configured for receiving incidental heat from said appliance element which produces said incidental heat, upon receiving said electrical supply, and thereby, at least in part, heats said volatilizable chemical to controllably emit said volatilized chemical.

7. A method of using a chemical delivery assembly, said method comprising the steps of:
providing an appliance including at least one of an electrical supply and an appliance element, the chemical delivery assembly including a volatilizable chemical and being carried by and coupled with said appliance;
heating said volatilizable chemical using at least one of said electrical supply and said appliance element; and
controllably emitting a volatilized chemical from said volatilizable chemical.

8. The method of claim 7, wherein the appliance is at least one of a laundry washer, a laundry dryer, a dishwasher, a refrigerator, an oven, and a television.

9. The method of claim 7, further including the step of supplying said electrical supply to said chemical delivery assembly, wherein said chemical delivery assembly is an air freshener assembly and said volatilizable chemical is formed as a fragrance gel, said air freshener assembly including a housing member attached to said appliance, a fragrance tray slidably received by said housing member, and said fragrance gel carried by said fragrance tray.

10. The method of claim 9, wherein said volatilized chemical is a fragrance and said air freshener assembly includes a printed circuit board and at least one heating element, said printed circuit board coupled with said housing member, said at least one heating element mounted on said printed circuit board under said fragrance tray and heating, upon said at least one heating element receiving and thereby being heated using said electrical supply, said fragrance gel to controllably emit said fragrance.

11. The method of claim 7, wherein said appliance element is a heater which produces heat upon receiving said electrical supply, said heater heating said volatilizable chemical to controllably emit said volatilized chemical.

12. The method of claim 7, wherein said appliance element produces, upon receiving said electrical supply, incidental heat which, at least in part, heats said volatilizable chemical in order to controllably emit said volatilized chemical.

13. The method of claim 7, wherein said volatilized chemical is provided internally to said appliance.
